(19) United States
(12) Patent Application Publication
(10) Pub. No.: US 2024/0168149 A1
(43) Pub. Date: May 23, 2024

(54) Localization and Health Monitoring

(71) Applicant: Google LLC, Mountain View, CA (US)

(72) Inventors: Aline Eid, Atlanta, GA (US); Jiang Zhu, Cupertino, CA (US); Luzhou Xu, San Diego, CA (US); Jimmy Georges Donald Hester, Atlanta, GA (US)

(73) Assignee: Google LLC, Mountain View, CA (US)

(21) Appl. No.: 18/552,622

(22) PCT Filed: Mar. 30, 2021
(86) PCT No.: PCT/US2021/024882

§ 371 Date: Sep. 26, 2023

(51) Int. Cl. G01S 13/56; G01S 7/41

(52) U.S. Cl. CPC G01S 13/56; G01S 7/415

(57) ABSTRACT

Techniques and apparatuses are described that implement localization and health monitoring. With the use of multiple backscatter tags, a reader can be implemented with a single antenna and have a relatively small footprint compared to other systems that utilize antenna arrays or multiple readers. Also, the backscatter tags can be implemented as passive devices and located at fixed positions. In this way, a single power source can be provided at the reader, and target angular resolutions can be realized without the additional mechanical complexities associated with other moving systems. Additionally, the backscatter tags can be implemented using relatively low-cost commercial off-the-shelf hardware. As such, performance of the reader can be readily customized with the purchase of additional backscatter tags.

### BACKGROUND

A health-monitoring device can help a user improve or maintain their health by measuring and reporting the user's vital signs. With this information, the health-monitoring device can evaluate a user's progress towards a fitness goal or detect an anomaly for early disease diagnosis and prevention. Some health-monitoring devices, however, are obtrusive and require contact with the user's skin to accurately measure the user's vital signs. This may make it cumbersome for the user to use while performing other activities or impede actions of nurses or doctors that are tending to the user.

### SUMMARY

Techniques and apparatuses are described that implement localization and health-monitoring using a reader and as few as two backscatter tags. With the use of multiple backscatter tags, a reader can be implemented with a single antenna and have a relatively small footprint compared to other systems that utilize antenna arrays. Also, the backscatter tags can be implemented as passive devices and located at fixed positions. In this way, a single power source can be provided at the reader, and target angular resolutions can be realized without the additional mechanical complexities associated with other moving systems. Additionally, the backscatter tags can be implemented using relatively low-cost commercial off-the-shelf hardware. As such, performance of the reader can be readily customized with the purchase of additional backscatter tags.

Aspects described below include a method performed by a reader for localization and health monitoring. The method includes transmitting a radio-frequency signal using an antenna of the reader. The radio-frequency signal interacts with two backscatter tags to form two backscatter signals. The two backscatter signals are reflected off of a person. The method also includes receiving the two backscatter signals reflected off of the person using the antenna. The method additionally includes determining, based on the two backscatter signals, a location of the person and a vital sign of the person.

Aspects described below also include a reader comprising at least one antenna. The reader is configured to perform any of the described methods.

Aspects described below include a system comprising at least two backscatter tags and a reader. The reader comprises at least one antenna and is configured to perform any of the described methods.

Aspects described below also include a system with means for performing localization and health monitoring.

### BRIEF DESCRIPTION OF DRAWINGS

Apparatuses for and techniques implementing localization and health monitoring are described with reference to the following drawings. The same numbers are used throughout the drawings to reference like features and components:

FIG. 1 illustrates example environments in which localization and health monitoring can be performed;

FIG. 2 illustrates an example implementation of a localization and health-monitoring system;

FIG. 3 illustrates example operations of a reader and multiple backscatter tags for localization and health monitoring;

FIG. 4 illustrates an example configuration of multiple backscatter tags for localization and health monitoring;

FIG. 5 illustrates an example scheme implemented by a reader for localization and health monitoring;

FIG. 6 illustrates an example method for performing operations of a reader capable of performing localization and health monitoring using multiple backscatter tags; and

FIG. 7 illustrates an example computing system embodying, or in which techniques may be implemented that enable use of, a reader capable of performing localization and health monitoring using multiple backscatter tags.

### DETAILED DESCRIPTION

### Overview

A health-monitoring device can help a user improve or maintain their health by measuring and reporting the user's vital signs. With this information, the health-monitoring device can evaluate a user's progress towards a fitness goal or detect an anomaly for early disease diagnosis and prevention. Some health-monitoring devices, however, are obtrusive and require contact with the user's skin to accurately measure the user's vital signs. This may make it cumbersome for the user to use while performing other activities or impede actions of nurses or doctors that are tending to the user.

To address this problem, some radio-frequency technologies can provide contactless health monitoring. For example, some radar systems can measure vital signs of one or more people. However, a radar system is a dedicated, complex system, which can be expensive. Also, to achieve sufficient angular resolutions for distinguishing between multiple people, the radar system can utilize an antenna array, which can increase a footprint and cost of the radar system. Alternatively, the radar system can use techniques such as synthetic-aperture radar (SAR) to realize target angular resolutions. Synthetic-aperture radar, however, relies on motion, which can increase a mechanical complexity of the radar system.

Some radio-frequency identification (RFID) systems can provide contactless health monitoring. Without the ability to provide localization, however, it can be challenging for these radio-frequency identification systems to operate in a multi-user environment. Other radio-frequency identification systems can provide localization using multiple readers and a large quantity of radio-frequency identification tags (e.g., approximately 30 or more). However, these systems may not be designed to operate in non-ideal environments (e.g., environments with multipath and clutter) or provide contactless health monitoring. Furthermore, the large quantity of radio-frequency identification tags can increase the installation complexity of the radio-frequency identification system.

In contrast, this document describes techniques and systems that implement localization and health monitoring using a reader and as few as two backscatter tags. With the use of multiple backscatter tags, the reader can be implemented with a single antenna and have a relatively small footprint compared to other radar systems that utilize antenna arrays or other radio-frequency identification systems that utilize multiple readers. Also, the backscatter tags can be implemented as passive devices and can be located at fixed positions. In this way, a single power source can be provided at the reader, and target angular resolutions can be realized without the additional mechanical complexities associated with other moving systems. The single power source and stationary components can make it easier to install the reader and the backscatter tags compared to other systems that utilize multiple power sources and/or moving components. Additionally, the backscatter tags can be implemented using relatively low-cost commercial off-the-shelf hardware. As such, performance of the reader can be readily customized with the purchase of additional backscatter tags.

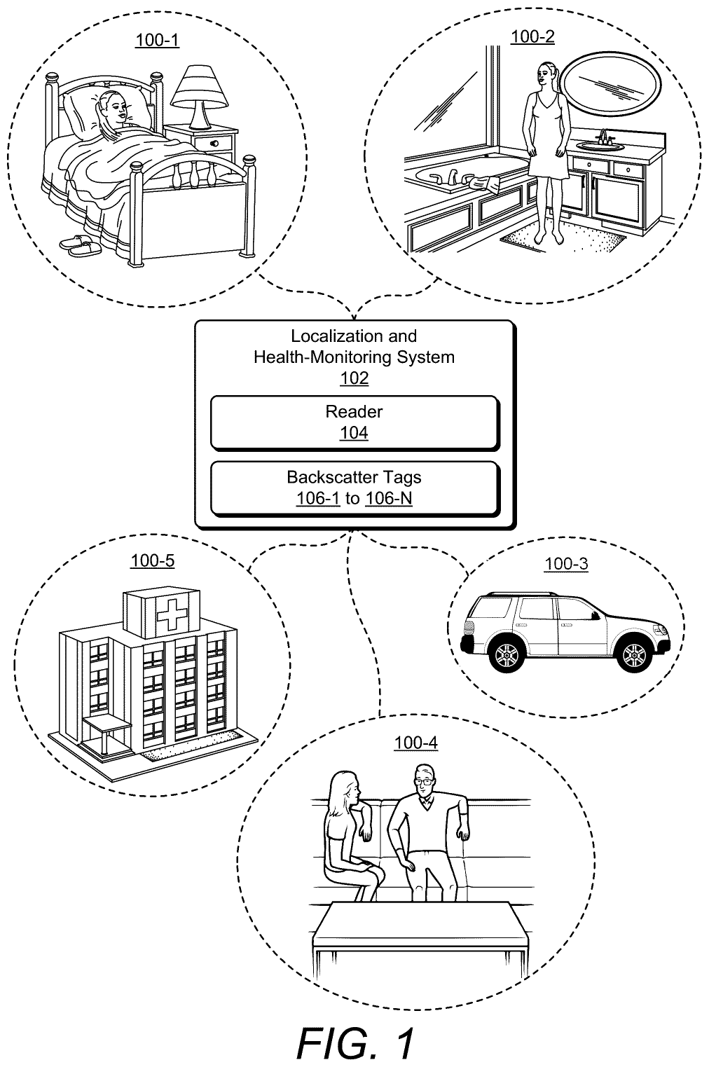

FIG. 1 illustrates example environments in which localization and health monitoring can be performed. In particular, a localization and health-monitoring system 102 provides contactless (non-contact) localization and health monitoring for one or more people. In some implementations, the localization and health-monitoring system 102 can provide the user information about a measured vital sign. Example vital signs can include a breathing rate or a heart rate. Additionally or alternatively, the localization and health-monitoring system 102 can alert the user or another entity (e.g., emergency services or a health professional) to an anomaly in the user's vital signs. In this way, the user can seek medical attention for early disease detection and prevention, or the other entity can provide emergency care, especially in situations in which the user becomes incapacitated.

The localization and health-monitoring system 102 can be installed in a variety of different environments, including those that may include multipath and clutter. Example environments can include glass, windows, concrete walls, metal separators, chairs, or screens (e.g., a monitor or a television).

In example environments 100-1 and 100-2, the localization and health-monitoring system 102 is installed in a room of a building that is frequented by a user. Example rooms include a bedroom in environment 100-1 and a bathroom in environment 100-2. The localization and health-monitoring system 102 can also be installed in other types of rooms, including an office or work room, or a cubicle within an office space.

In the environment 100-1, the localization and health-monitoring system 102 can monitor a user's vital sign as the user sleeps. In some cases, a quality of the user's sleep can be measured based on the vital sign information provided by the localization and health-monitoring system 102. Additionally or alternatively, the localization and health-monitoring system 102 can detect occurrences of sleep apnea that disrupt the user's breathing. With knowledge of the frequency and duration of the sleep apnea, the user may choose to seek medical treatment.

In the environment 100-2, the localization and health-monitoring system 102 can evaluate the user's vital sign while they are present and using the facilities. In some implementations, the localization and health-monitoring system 102 can detect that a stroke or heart attack occurred and automatically notify a family member or emergency services to assist the user.

In some environments, the localization and health-monitoring system 102 is installed at a location that is frequented by multiple people. For example, the localization and health-monitoring system 102 can be installed within a vehicle, a living room, or a medical facility (e.g., within awaiting room or a patient's room), as illustrated in example environments 100-3, 100-4, and 100-5, respectively. Other example environments can include a gym or a restaurant.

Within the medical facility of environment 100-5, the localization and health-monitoring system 102 can detect that a particular patient within a waiting room is having trouble breathing or has an abnormal heart rate, and alert the staff to the patient's condition. As another example, the localization and health-monitoring system 102 can detect if a particular guest at a restaurant is experiencing an allergic reaction or choking, and send staff to that diner's table to assist the guest. In other situations, such as at the gym, the localization and health-monitoring system 102 can provide an individual with their vital sign measurements during an exercise routine.

To provide localization and health monitoring, the localization and health-monitoring system 102 includes at least one reader 104 (e.g., one reader 104) and at least two backscatter tags 106-1 to 106-N, where N represents a positive integer greater than or equal to two. The reader 104 can be implemented as a radio-frequency identification reader, and the backscatter tags 106-1 to 106-N can be implemented as radio-frequency identification tags. In example implementations, the quantity of backscatter tags 106-1 to 106-N can be equal to two, four, eight, or more. In general, increasing the quantity of backscatter tags (e.g., increasing N) increases an angular resolution and accuracy of the localization and health-monitoring system 102. With two or more backscatter tags 106-1 to 106-N, the localization and health-monitoring system 102 can distinguish between different people that are less than half a meter apart (e.g., less than approximately 50 centimeters apart or less than approximately 25 centimeters apart).

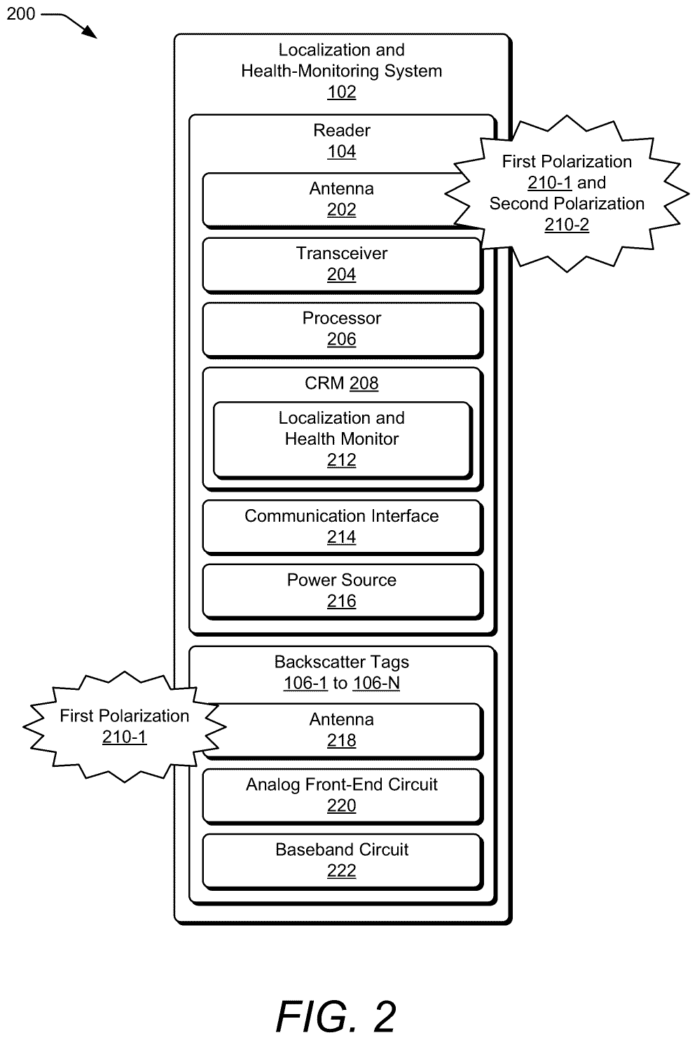

Some localization and health-monitoring systems 102 can be integrated as part of a smart home system. With the ability to operate with fewer components compared to other systems, the localization and health-monitoring system 102 can be relatively easy for a user to install along one or more walls of a residential room. In an example situation, the localization and health-monitoring system 102 can monitor a 10 foot by 10 foot room using four backscatter tags 106 installed across one wall of the room. In this case, the localization and health-monitoring system 102 can measure both a vital sign and location of the user while the user is present within the room. To improve the location measurement, the user can install four additional backscatter tags 106 on another wall that is orthogonal to the wall with the other backscatter tags 106. Because the backscatter tags 106 can operate without a dedicated power source, the installation process can be less complicated compared to other systems and the user can have additional flexibility in positioning the backscatter tags 106. At a later time, the user can improve the resolution and capability of the localization and health-monitoring system 102 by installing more than four backscatter tags 106 on a wall. With additional resolution, the localization and health-monitoring system 102 can support other operations, such as gesture recognition, fall detection, or collision avoidance. The localization and health-monitoring system 102 is further described with respect to FIG. 2.

FIG. 2 illustrates an example localization and health-monitoring system 102. In general, the localization and health-monitoring system 102 uses radio-frequency signals to evaluate signal path propagations within a backscatter environment. In the depicted configuration, the localization and health-monitoring system 102 includes the reader 104 and the backscatter tags 106-1 to 106-N. The reader 104 generates the radio-frequency signals. The backscatter tags 106-1 to 106-N interact with the radio-frequency signals to generate the backscatter environment. In general, the backscatter tags 106-1 to 106-N operate as a quasi-virtual antenna array of the reader 104 to enable localization and health monitoring.

The reader 104 includes at least one antenna 202, at least one transceiver 204, at least one processor 206, and at least one computer-readable storage medium 208 (CRM 208). The antenna 202 can be implemented using a patch antenna (e.g., a microstrip antenna), a dipole antenna, or a helical antenna. In an example implementation, the antenna 202 can have a gain of approximately 9 dBi or more.

In some implementations, the antenna 202 transmits and receives using orthogonal polarizations. For example, the antenna 202 can transmit radio-frequency signals using a first polarization 210-1 and receive radio-frequency signals using a second polarization 210-2, which is orthogonal to the first polarization 210-1. The first polarization 210-1 and the second polarization 210-2 can be circular polarizations. For example, the first polarization 210-1 can be a left-hand circular polarization (LHCP), and the second polarization 210-2 can be a right-hand circular polarization (RHCP). In another example, the first polarization 210-1 can be the RHCP, and the second polarization 210-2 can be the LHCP.

The antenna 202 can be implemented using a single antenna. For example, the antenna 202 can include a dual linearly-polarized patch antenna. The patch antenna can have a square (e.g., rectangular) or circular shape. To generate the orthogonal circular polarizations, two feeds of the patch antenna can be coupled to two ports of a quadrature hybrid coupler, which have a 90 degree phase difference. The two feeds of the patch antenna can produce an electric field that varies in two orthogonal dimensions and results in the transmission and reception of circularly-polarized signals of opposite handedness.

Alternatively, the antenna 202 can be implemented using at least two antennas. For example, a first antenna can transmit the radio-frequency signals with the first polarization 210-1, and a second antenna can receive the radio-frequency signals with the second polarization 210-2.

The transceiver 204 is coupled to the antenna 202 and includes circuitry and logic for transmitting and receiving the radio-frequency signals via the antenna 202. Components of the transceiver 204 can include amplifiers, oscillators, and so forth. The transceiver 204 can also include logic to perform in-phase/quadrature (I/Q) operations, such as modulation or demodulation. In an example implementation, the transceiver 204 can have a transmit power of approximately 30 dBm or more and a sensitivity of at least −84 dBm.

The processor 206 executes instructions that are stored within the CRM 208. The CRM 208 enables persistent and/or non-transitory data storage (i.e., in contrast to mere signal transmission). The processor 206 can be integrated within the transceiver 204 or implemented on a separate integrated circuit. The CRM 208 includes a localization and health monitor 212. The localization and health monitor 212 uses holography and beamforming to measure locations and vital signs of one or more people.

The reader 104 can optionally include a communication interface 214 and a power source 216. The communication interface 214 can communicate data over wired, wireless, or optical networks. The power source 216 can be an internal power source, such as a battery. In other implementations, the reader 104 can include a connection to an external power source, such as a solar panel, an external battery, or a power outlet.

The backscatter tags 106-1 to 106-N can each include at least one antenna 218, at least one analog front-end circuit 220, and at least one baseband circuit 222. The backscatter tags 106-1 to 106-N can be implemented as passive devices, which are powered by incoming radio-frequency signals. In this way, the backscatter tags 106-1 to 106-N can be powered wirelessly and can operate without a dedicated power source.

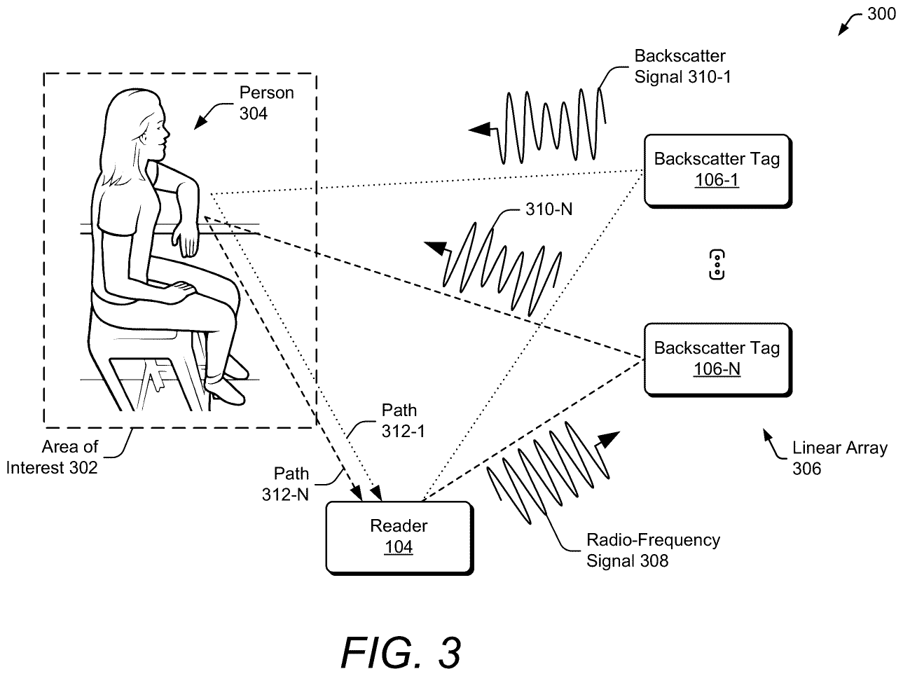

The antenna 218 can have the first polarization 210-1. In this way, the antenna 218 is co-polarized with the transmission of the antenna 202 and cross-polarized with the reception of the antenna 202. This enables the reader 104 to attenuate radio-frequency signals that propagate from the backscatter tags 106-1 to 106-N along a line-of-sight propagation path, as further described with respect to FIG. 3.

The analog front-end circuit 220 includes circuitry and logic for transmitting and receiving radio-frequency signals via the antenna 218. The analog front-end circuit 220 can include logic to perform in-phase/quadrature (I/Q) operations, such as modulation or demodulation. Additionally, the analog front-end circuit 220 can harvest energy from an incoming radio-frequency signal for power.

The baseband circuit 222 can process data that is demodulated by the analog front-end circuit 220 or provide data to the analog front-end circuit 220 for modulation. As an example, the baseband circuit 222 can store an identification number of the corresponding backscatter tag 106-1 to 106-N and provide the identification number to the analog front-end circuit 220. The analog front-end circuit 220 can modulate a radio-frequency signal based on the identification number to enable the reader 104 to associate the radio-frequency signal as being generated by the corresponding backscatter tag 106-1 to 106-N. Example modulations can include amplitude modulation, phase modulation, and/or frequency modulation.

A frequency spectrum (e.g., range of frequencies) that the reader 104 and the backscatter tags 106-1 to 106-N use can include radio frequencies (e.g., frequencies between approximately 30 hertz (Hz) and 300 gigahertz (GHz)). In some implementations, the frequency spectrum includes ultra-high frequencies (UHF) (e.g., frequencies between approximately 300 and 3000 megahertz (MHz)). In this case, the reader 104 can be implemented as an ultra-high-frequency (UHF) radio-frequency identification (RFID) reader, and the backscatter tags 106-1 to 106-N can be implemented as UHF RFID tags. As an example, the UHF RFID reader and UHF RFID tags can operate with frequencies that are between approximately 902 and 928 MHz. To support the UHF frequencies, dimensions of the antenna 202 of the reader 104 and the antennas 218 of the backscatter tags 106-1 to 106-N can be at least 10 millimeters (mm) by 10 mm. Some example implementations have dimensions are approximately 2 centimeters (cm) by 2 cm.

Additionally or alternatively, the frequency spectrum of the reader 104 and backscatter tags 106-1 to 106-N can include extremely-high frequencies (EHF) associated with millimeter wavelengths. Example frequencies can include those between approximately 30 and 300 GHz (e.g., approximately 60 GHz). To support the EHF frequencies, dimensions of the antenna 202 of the reader 104 and the antennas 218 of the backscatter tags 106-1 to 106-N can be at least 0.2 mm by 0.2 mm. Some example implementations are approximately 2 mm by 2 mm.

To provide localization and health monitoring for a 10 foot by 10 foot room, the localization and health-monitoring system 102 can include UHF or EHF backscatter tags 106-1 to 106-N that are installed along at least one wall of the room. As an example, the backscatter tags 106-1 to 106-N can be separated by approximately half a wavelength or more. For example, four backscatter tags 106-1 to 106-N that operate at approximately 60 GHz can be spaced approximately 0.25 cm apart or more across the wall. Operations of the reader 104 and the backscatter tags 106-1 to 106-N are further described with respect to FIG. 3.

FIG. 3 illustrates example operation of the reader 104 and the backscatter tags 106-1 to 106-N. In an environment 300, the reader 104 and the backscatter tags 106-1 to 106-N are positioned proximate to an area of interest 302. The area of interest 302 represents a region in which one or more people, including person 304, are likely to be located. The localization and health-monitoring system 102 measures the location and vital signs of people within the area of interest 302. In some implementations, the area of interest 302 can include a region in which angular ambiguities associated with the localization and health-monitoring system 102 are reduced in quantity relative to other regions outside of the area of interest 302.

In the environment 100-1, the area of interest 302 can include the bed. In the environment 100-2, the area of interest 302 can include a region next to a sink. As another example, the area of interest 302 can include various positions at a desk. In some case, a size of the area of interest 302 can be on the order of ten square meters, such as approximately 12 square meters. In other cases, the size of the area of interest 302 can be twenty square meters or more to encompass a significant portion of a room, such as a waiting room of the medical institution in environment 100-5 or an exercise room of a gym.

In the depicted configuration, the backscatter tags 106-1 to 106-N and the reader 104 are arranged on adjacent sides of the area of interest 302. For example, the backscatter tags 106-1 to 106-N are positioned on a right side of the area of interest 302 and the reader 104 is positioned on a bottom side of the area of interest 302. If the quantity of backscatter tags (N) is greater than two, the backscatter tags 106-1 to 106-N can be arranged in a line to form a linear array 306. A spacing between the backscatter tags 106-1 to 106-N can be larger than half a wavelength to reduce coupling between the backscatter tags 106-1 to 106-N. The wavelength can refer to a wavelength associated with a frequency spectrum of the localization and health-monitoring system 102. As an example, the spacing between the backscatter tags 106-1 to 106-N can be approximately three-quarters of a wavelength. In general, the spacing can be chosen to reduce the quantity of sidelobes within the area of interest 302.

During operation, the reader 104 transmits a radio-frequency signal 308. The radio-frequency signal 308 can be a continuous-wave signal and can have the first polarization 210-1. In some implementations, the reader 104 uses a frequency-hopping spread-spectrum (FHSS) technique to transmit the radio-frequency signal 308. As a result, the radio-frequency signal 308 can have a frequency that changes between consecutive time periods. For example, the radio-frequency signal 308 can have a frequency that hops approximately every ten seconds and cycles between frequencies associated with ten different frequency channels or more.

At separate times, the backscatter tags 106-1 to 106-N receive the radio-frequency signal 308 and generate respective backscatter signals 310-1 to 310-N based on the radio-frequency signal 308. In this way, the backscatter tags 106-1 to 106-N interact with the radio-frequency signal 308 to create a backscatter environment for localization and health monitoring. In some implementations, the backscatter signals 310-1 to 310-N represent modified versions of the radio-frequency signal 308. For example, the backscatter tags 106-1 to 106-N can modulate data, such as a corresponding identification number, onto the backscatter signals 310-1 to 310-N. The backscatter tags 106-1 to 106-N can generate the backscatter signals 310-1 to 310-N with the first polarization 210-1.

The backscatter signals 310-1 to 310-N impinge on the person 304 within the area of interest 302 (e.g., reflect off of the person 304) and propagate back towards the reader 104. Due to the reflection, a handedness of the reflected backscatter signals 310-1 to 310-N is reversely polarized. For example, a backscatter signal 310 with the first polarization 210-1 is reflected with the second polarization 210-2.

The reflected backscatter signals 310-1 to 310-N can also have frequencies that differ from the transmitted backscatter signals 310-1 to 310-N due to the Doppler effect. More specifically, the reflected backscatter signals 310-1 to 310-N can include a frequency component associated with the person's 304 vital sign, such as the person 304's breathing rate or heart rate.

The reader 104 receives the reflected backscatter signals 310-1 to 310-N. In some implementations, the reader 104 receives the reflected backscatter signals 310-1 to 310-N using a same polarization as the reflected backscatter signals 310-1 to 310-N. By transmitting and receiving with opposite handedness, the reflected backscatter signals 310-1 to 310-N can have a higher signal strength at the reader 104 compared to other signals within the environment 300, including the radio-frequency signal 308 or non-reflected versions of the backscatter signals 310-1 to 310-N. The non-reflected versions of the backscatter signals 310-1 to 310-N can propagate along a line-of-sight between the reader 104 and the corresponding backscatter tags 106-1 to 106-N. This polarization mismatch enables the reader 104 to operate at farther distances from the backscatter tags 106-1 to 106-N and the area of interest 302.

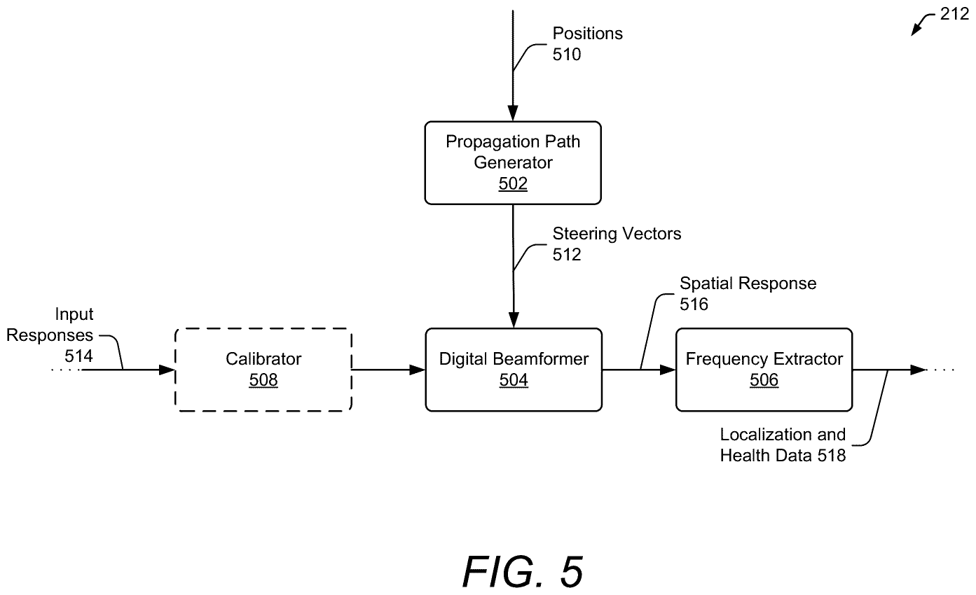

Propagation paths 312-1 to 312-N (paths 312-1 to 312-N) characterize the propagation of the radio-frequency signal 308 from the reader 104 to the individual backscatter tags 106-1 to 106-N, the propagation of the backscatter signals 310-1 to 310-N from the corresponding backscatter tags 106-1 to 106-N to the area of interest 302, and the propagation of reflected versions of the backscatter signals 310-1 to 310-N from the area of interest 302 to the reader 104. Phases of the reflected backscatter signals 310-1 to 310-N received at the reader 104 can vary based on the lengths (e.g., distances) of the propagation paths 312-1 to 312-N. These phases can provide information about the location of the person 304 within the area of interest 302, as further described with respect to FIG. 5.

A proximity of the reader 104 and backscatter tags 106-1 to 106-N to the area of interest 302 can be based on the transmit power and sensitivity of the reader 104. In general, the positions and orientations of the reader 104 and backscatter tags 106-1 to 106-N is sufficient for the radio-frequency signal 308 to propagate to the backscatter tags 106-1 to 106-N and is sufficient for the reflected versions of the backscatter signals 310-1 to 310-N to be received at the reader 104.

If an antenna pattern of the antenna 202 of the reader 104 has a main lobe, the main lobe can be oriented towards the backscatter tags 106-1 to 106-N and the area of interest 302. If the antenna 202 includes a separate transmit antenna and a separate receive antenna, a main lobe of the transmit antenna can be directed towards the backscatter tags 106-1 to 106-N and a main lobe of the receive antenna can be directed towards the area of interest 302. Likewise, main lobes of the backscatter tags 106-1 to 106-N can be directed towards the reader 104 and the area of interest 302.

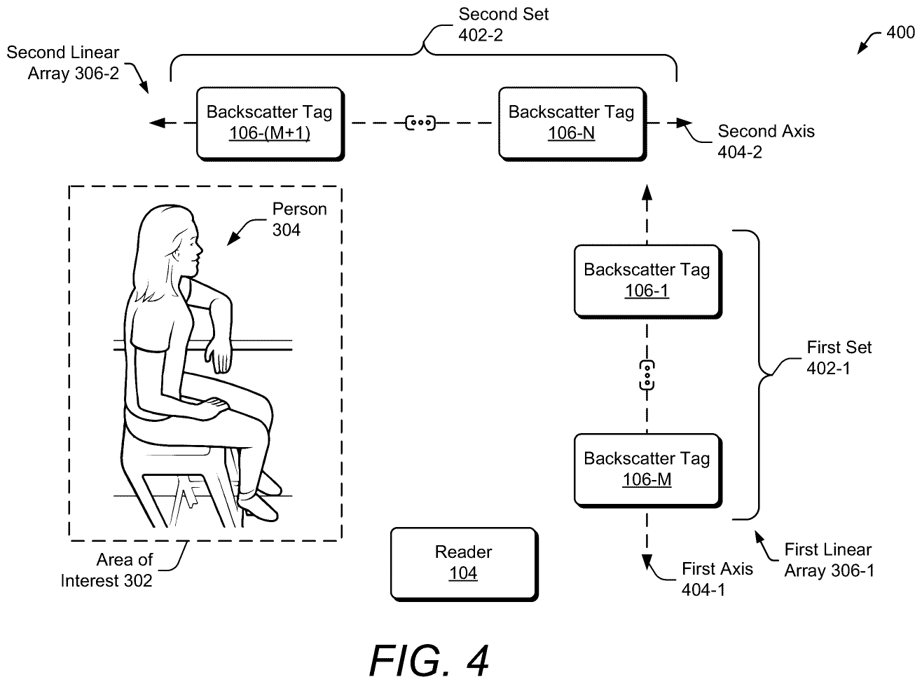

Using the linear array 306 of backscatter tags 106-1 to 106-N, the reader 104 can measure a direction-of-arrival (DOA) (or an angle-of-arrival (AOA)) of the reflected backscatter signals 310-1 to 310-N to determine a location of the person 304. Alternatively, another arrangement of the backscatter tags 106-1 to 106-N can enable the reader 104 to measure a multi-dimensional location of the person 304, as further described with respect to FIG. 4.

FIG. 4 illustrates another example configuration of the backscatter tags 106-1 to 106-N for localization and health monitoring. In the environment 400, the backscatter tags 106-1 to 106-N are divided into two sets. A first set 402-1 includes backscatter tags 106-1 to 106-M, where M represents a positive integer that is less than N. A second set 402-2 includes backscatter tags 106-(M+1) to 106-N. The first set 402-1 of backscatter tags are positioned along a first axis 404-1 to form a first linear array 306-1. The second set 402-2 of backscatter tags are positioned along a second axis 404-2 to form a second linear array 306-2. The second axis 404-2 is approximately perpendicular to the first axis 404-1.

The quantity of backscatter tags within the first set 402-1 (M) can be equal to, greater than, or less than the quantity of backscatter tags within the second set 402-2 (N-M). In an example implementation, N can be equal to four, and M can be equal to two. In another example implementation, N can be equal to eight, and M can be equal to four.

In the depicted configuration, the first set 402-1 of backscatter tags, the second set 402-2 of backscatter tags, and the reader 104 are arranged on adjacent sides of the area of interest 302. For example, the reader 104 is positioned on a bottom side of the area of interest 302. The first set 402-1 of backscatter tags are positioned on a right side of the area of interest 302, which is adjacent to the bottom side. The second set 402-2 of backscatter tags are positioned on atop side of the area of interest 302, which is adjacent to the right side. A spacing between the backscatter tags within the first set 402-1 and second set 402-2 can be larger than half a wavelength to reduce coupling. As an example, the spacing between the backscatter tags 106-1 to 106-M within the first set 402-1 and the spacing between the backscatter tags 106-(M+1) to 106-N within the second set 402-2 can be approximately three-quarters of a wavelength.

Together, the first set 402-1 and second set 402-2 of backscatter tags form a two-dimensional array, which enables the reader 104 to measure a location of the person 304 across two dimensions associated with the first axis 404-1 and the second axis 404-2. With the backscatter tags 106-1 to 106-N forming a one-dimensional array, as shown in FIG. 3, or a multi-dimensional array, as shown in FIG. 4, the reader 104 can measure the vital sign and location of the person 304, as further described with respect to FIG. 5.

Although not explicitly shown, other implementations of the localization and health-monitoring system 102 can position the backscatter tags 106-1 to 106-N in other arrangements. For example, the backscatter tags 106-1 to 106-N can be positioned to form a three-dimensional array. As such, the reader 104 can measure a three-dimensional location of the person 304. Alternatively, the backscatter tags 106-1 to 106-N can be positioned in a distributed (non-linear) arrangement.

FIG. 5 illustrates an example scheme implemented by the reader 104 for localization and health monitoring. In the depicted configuration, the reader 104 implements the localization and health monitor 212, which includes a propagation path generator 502, a digital beamformer 504, and a frequency extractor 506. The localization and health monitor 212 can also optionally include a calibrator 508 if the reader 104 uses a FHSS technique to transmit the radio-frequency signal 308.

During initialization, the localization and health monitor 212 stores information about positions 510 of the antenna 202 of the reader 104 and the antennas 218 of the backscatter tags 106-1 to 106-N. The positions 510 can be relative positions based on a reference point, such as a center of the linear array 306 or the antenna 202 of the reader 104. This information can be provided by a user or automatically determined by the localization and health-monitoring system 102 using ranging techniques.

During operation, the propagation path generator 502 uses a propagation model to generate steering vectors 512 based on the positions 510. The steering vectors 512 can be determined based on resulting propagation phases, which are dependent upon the distances of modeled propagation paths and the wavelength of the radio-frequency signals. In other words, the steering vectors 512 represent a delay encountered due to propagation path differences.

The localization and health monitor 212 accepts, from the transceiver 204, input responses 514 associated with the received backscatter signals 310-1 to 310-N. The input responses 514 can be in a time domain or a frequency domain. In general, the input responses 514 include digital samples having amplitude information (e.g., received signal strength indicator (RSSI) information) and phase information.

Due to the operations of the backscatter tags 106-1 to 106-N occurring at different time intervals, the input responses 514 are generated in series for each of the backscatter signals 310-1 to 310-N. The localization and health monitor 212 can store the input responses 514 and proceed to the next operation once an input response 514 for each backscatter signal 310-1 to 310-N is available. The localization and health monitor 212 can detect this event by identifying the identification numbers provided by the backscatter signals 310-1 to 310-N. The input responses 514 can be provided to the calibrator 508 or the digital beamformer 504, depending on the implementation.

If the reader 104 uses an FHSS technique to transmit the radio-frequency signal 308, the calibrator 508 can calibrate the input responses 514 to reduce variations caused by the FHSS technique. In particular, the calibrator 508 can remove a medium value of the amplitude and phase associated with each input response 514. The calibrator 508 can also unwrap the phase within the input responses 514 to remove ambiguities associated with the phase being greater than or equal to 180 degrees.

The digital beamformer 504 accepts the steering vectors 512 and input responses 514 (or calibrated input responses 514 provided by the calibrator 508). Using conventional beamforming techniques or adaptive beamforming techniques, the digital beamformer 504 generates a spatial response 516 based on the steering vectors 512 and the input responses 514.

In an example implementation, the digital beamformer 504 implements a delay-and-sum beamformer. The delay-and-sum beamformer determines weights based on the steering vectors 512 (e.g., based on complex conjugates of the steering vectors 512). In this case, the weights are not derived based on the input responses 514. As such, the weights can be considered data-independent weights. The delay-and-sum beamformer applies the weights to the input responses 514 and performs a summation across the weighted input responses 514. The summation of the weighted input responses 514 produces a spatial response 516, which includes amplitude and phase information.

In another example implementation, the digital beamformer 504 implements a Capon beamformer (e.g., a minimum-variance distortionless-response (MVDR) beamformer). The Capon beamformer determines weights based on the steering vectors 512 and the input responses 514. In particular, the Capon beamformer calculates the weights based on covariance matrices of the input responses 514 and the steering vectors 512. The Capon beamformer applies the weights to the input responses 514 to generate the spatial response 516.

The spatial response 516 can include direction-of-arrival information for a one-dimensional array of backscatter tags 106-1 to 106-N or multi-dimensional information for a multi-dimensional array of backscatter tags 106-1 to 106-N. By analyzing the spatial response 516, the localization and health monitor 212 can determine a location of the person 304.

The frequency extractor 506 can extract the frequencies associated with different spatial locations within the spatial response 516. By analyzing the frequencies at a location corresponding to the person 304, the frequency extractor 506 can determine a vital sign of the person 304, including a breathing rate and/or a heart rate. In some situations, the frequency extractor 506 can measure the vital sign of the person 304 based on the frequency with a highest amplitude. The frequency extractor 506 generates localization and health data 518, which includes the measured location and measured vital sign of the person 304. If multiple people are present within the area of interest 302, the localization and health data 518 can include locations and vital signs for each person.

In general, the localization and health monitor 212 combines holography and beamforming techniques to localize and evaluate the health of the person 304. The techniques of holography enable the reader 104 to generate a representative digital signature of the person 304 based on the positions 510 of the reader 104 and the backscatter tags 106-1 to 106-N. In particular, the propagation path generator 502 and the digital beamformer 504 enable the localization and health monitor 212 to correlate the input responses 514 with the reference steering vectors 512 to localize and monitor the health of the person 304.

The localization and health monitor 212 can provide the localization and health data 518 to the communication interface 214, which can perform an action to convey this information to the person 304 or another entity. For example, the communication interface 214 can display this information or communicate this information to another device.

In example implementations, the localization and health-monitoring system 102 can measure breathing rates between approximately 4.5 and 60 beats-per-minute (bmp) with errors of approximately 2 bmp or less (e.g., less than 1 bmp). Depending on the quantity of backscatter tags (N) and the arrangement of the backscatter tags 106-1 to 106-N, the localization and health-monitoring system 102 can achieve various location accuracies. In an example implementation in which the localization and health-monitoring system 102 includes four backscatter tags 106 arranged to form the linear array 306 of FIG. 3, the localization and health-monitoring system 102 can have a direction-of-arrival error less than approximately ten degrees for detecting one person 304. In another example implementation in which the localization and health-monitoring system 102 includes eight backscatter tags 106 arranged to form the linear arrays 306-1 and 306-2 of FIG. 4, the localization and health-monitoring system 102 can have a location error of less than approximately twenty centimeters along the first axis 404-1 or the second axis 404-2.

### Example Method

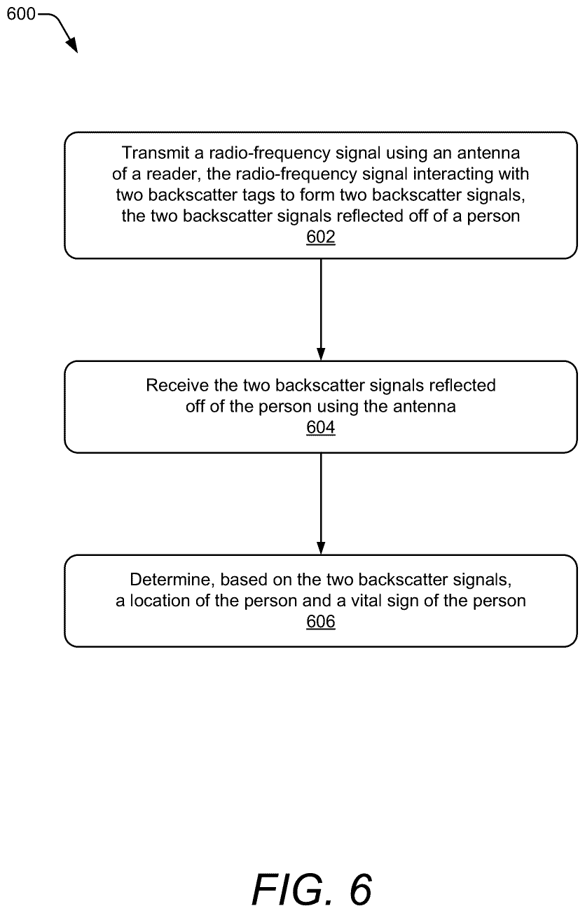

FIG. 6 depicts an example method 600 for performing operations of localization and health monitoring. Method 600 is shown as sets of operations (or acts) performed but not necessarily limited to the order or combinations in which the operations are shown herein. Further, any of one or more of the operations may be repeated, combined, reorganized, or linked to provide a wide array of additional and/or alternate methods. In portions of the following discussion, reference may be made to the environment 100-1 to 100-5 of FIG. 1, and entities detailed in FIG. 2 or 3, reference to which is made for example only. The techniques are not limited to performance by one entity or multiple entities operating on one device.

At 602, a radio-frequency signal is transmitted using an antenna of a reader. The radio-frequency signal interacts with two backscatter tags to form two backscatter signals. The two backscatter signals are reflected off of a person. For example, the reader 104 transmits the radio-frequency signal 308 using the antenna 202, as shown in FIG. 3. In some implementations, the reader 104 transmits the radio-frequency signal 308 with the first polarization 210-1, such as a first circular polarization. The reader 104 can also transmit the radio-frequency signal 308 using a frequency-hopping spread-spectrum technique, which changes the frequency of the radio-frequency signal 308 between consecutive time intervals.

The radio-frequency signal 308 interacts with the two backscatter tags 106-1 to 106-N to form the backscatter signals 310-1 to 310-N, as shown in FIG. 3. The backscatter signals 310-1 to 310-N can have a same polarization as the radio-frequency signal 308. The backscatter signals 310-1 to 310-N can be formed at different times.

The backscatter signals 310-1 to 310-N are reflected off of the person 304, as shown in FIG. 3. Due to the reflection, the reflected backscatter signals 310-1 to 310-N can have an orthogonal polarization relative to the radio-frequency signal 308. For example, the reflected backscatter signals 310-1 to 310-N can have the second polarization 210-2. The reflected backscatter signals 310-1 to 310-N can include frequency components based on a vital sign of the person 304. Also, the reflected backscatter signals 310-1 to 310-N can include phases that are based on a location of the person 304, a position of the reader 104, and positions of the corresponding backscatter tags 106-1 to 106-N.

At 604, the two backscatter signals reflected by the at least one person are received using the antenna. For example, the reader 104 receives the reflected backscatter signals 310-1 to 310-N using the antenna 202. The reader 104 can receive the reflected backscatter signals 310-1 to 310-N using the second polarization 210-2, which is orthogonal to the first polarization 210-1.

At 606, a location of the person and a vital sign of the person are determined based on the two backscatter signals. For example, the reader 104 determines the location of the person 304 and the vital sign of the person 304 based on the backscatter signals 310-1 to 310-N. The location can include a direction-of-arrival or a multi-dimensional location (e.g., X and Y coordinates, or X, Y, and Z coordinates) of the person 304. The vital sign can include a breathing rate and/or a heart rate of the person 304. The reader 104 can use holography and beamforming techniques to measure the position of the person 304 and the vital sign of the person 304, as further described with respect to FIG. 5.

### Example Computing System

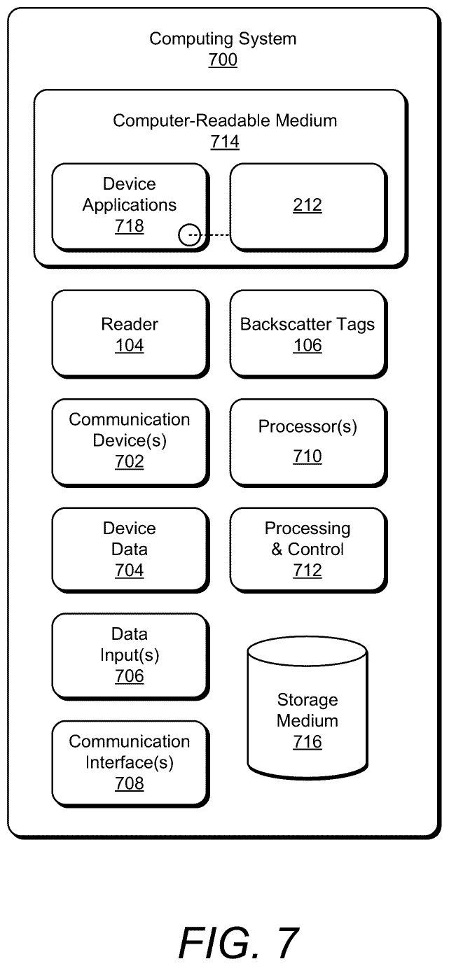

FIG. 7 illustrates various components of an example computing system 700 that can be implemented as any type of computing system to perform localization and health monitoring. The computing system 700 includes the reader 104 and the backscatter tags 106-1 to 106-N of FIG. 2. The computing system 700 also includes communication devices 702 that enable wired and/or wireless communication of device data 704. The device data 704 or other device content can include localization and health data 518 generated by the reader 104. The computing system 700 includes one or more data inputs 706 via which any type of data and/or inputs can be received, including the positions 510 of the reader 104 and the backscatter tags 106-1 to 106-N.

The computing system 700 also includes communication interfaces 708, which can be implemented as any one or more of a serial and/or parallel interface, a wireless interface, any type of network interface, a modem, and as any other type of communication interface. The communication interfaces 708 provide a connection and/or communication links between the computing system 700 and a communication network by which other electronic, computing, and communication devices communicate data with the computing system 700.

The computing system 700 includes one or more processors 710 (e.g., any of microprocessors, controllers, and the like), which process various computer-executable instructions to control the operation of the computing system 700 and to enable techniques for, or in which can be embodied, localization and health monitoring. Alternatively or in addition, the computing system 700 can be implemented with any one or combination of hardware, firmware, or fixed logic circuitry that is implemented in connection with processing and control circuits which are generally identified at 712. Although not shown, the computing system 700 can include a system bus or data transfer system that couples the various components within the system. A system bus can include any one or combination of different bus structures, such as a memory bus or memory controller, a peripheral bus, a universal serial bus, and/or a processor or local bus that utilizes any of a variety of bus architectures.

The computing system 700 also includes a computer-readable medium 714, such as one or more memory devices that enable persistent and/or non-transitory data storage (i.e., in contrast to mere signal transmission), examples of which include random access memory (RAM), non-volatile memory (e.g., any one or more of a read-only memory (ROM), flash memory, EPROM, EEPROM, etc.), and a disk storage device. The disk storage device may be implemented as any type of magnetic or optical storage device, such as a hard disk drive, a recordable and/or rewriteable compact disc (CD), any type of a digital versatile disc (DVD), and the like. The computing system 700 can also include a mass storage media device (storage media) 716.

The computer-readable medium 714 provides data storage mechanisms to store the device data 704, as well as various device applications 718 and any other types of information and/or data related to operational aspects of the computing system 700. For example, an operating system can be maintained as a computer application with the computer-readable medium 714 and executed on the processors 710. The device applications 718 may include a device manager, such as any form of a control application, software application, signal-processing and control module, code that is native to a particular device, a hardware abstraction layer for a particular device, and so on.

The device applications 718 also include any system components, engines, or managers to implement localization and health monitoring. In this example, the device applications 718 includes the localization and health monitor 212 of FIG. 2.

Although described with respect to health monitoring, the localization and health-monitoring system 102 can be designed to perform other operations in addition to or instead of health monitoring. In some implementations, the localization and health-monitoring system 102 can support other applications, including gesture recognition, fall detection, or collision avoidance.

### CONCLUSION

Although techniques using, and apparatuses including, localization and health monitoring have been described in language specific to features and/or methods, it is to be understood that the subject of the appended claims is not necessarily limited to the specific features or methods described. Rather, the specific features and methods are disclosed as example implementations of localization and health monitoring.

Some examples are described below.

Example 1: A method performed by a reader, the method comprising:
transmitting a radio-frequency signal using an antenna of the reader, the radio-frequency signal interacting with two backscatter tags to form two backscatter signals, the two backscatter signals reflected off of a person;
receiving the two backscatter signals reflected off of the person using the antenna; and
determining, based on the two backscatter signals, a location of the person and a vital sign of the person.

Example 2: The method of example 1, wherein:
the two backscatter signals are reflected off of another person; and
the determining comprises determining a location of the other person and a vital sign of the other person.

Example 3: The method of example 1 or 2, wherein,
the transmitting of the radio-frequency signal comprises transmitting the radio-frequency signal in a direction towards the two backscatter tags; and
the receiving of the two backscatter signals comprises receiving the two backscatter signals from a direction associated with an area of interest in which the person is located.

Example 4: The method of example 3, wherein the area of interest comprises:
a room in a building;
a bed;
a desk; or
a vehicle.

Example 5: The method of any preceding example, wherein:
the transmitting of the radio-frequency signal comprises transmitting the radio-frequency signal with a first polarization that is co-polarized with antennas of the backscatter tags; and
the receiving of the radio-frequency signal comprises receiving the radio-frequency signal with a second polarization that is cross-polarized with the antennas of the backscatter tags.

Example 6: The method of example 5, wherein:
the first polarization comprises a first circular polarization; and
the second polarization comprises a second circular polarization that is orthogonal to the first circular polarization.

Example 7: The method of example 6, wherein:
the first circular polarization comprises a left-hand circular polarization; and
the second circular polarization comprises a right-hand circular polarization.

Example 8: The method of any preceding example, wherein the vital sign comprises at least one of a breathing rate or a heart rate.

Example 9: The method of any preceding example, wherein:
the transmitting of the radio-frequency signal comprises transmitting the radio-frequency signal using a frequency-hopping spread-spectrum technique; and
the determining comprises calibrating input responses associated with the two backscatter signals to reduce variations caused by the frequency-hopping spread-spectrum technique.

Example 10: A reader comprising at least one antenna, the reader configured to perform any of the methods of claims 1 to 9.

Example 11: The reader of example 10, wherein the at least one antenna comprises:
a transmit antenna; and
a receive antenna.

Example 12: The reader of example 10, wherein the at least one antenna is configured to transmit and receive radio-frequency signals with orthogonal circular polarizations.

Example 13: A system comprising:
at least two backscatter tags; and
a reader comprising at least one antenna, the reader configured to perform any of the methods of claims 1 to 9.

Example 14: The system of example 13, wherein the at least two backscatter tags comprise at least four backscatter tags.

Example 15: The system of example 14, wherein an arrangement of the at least four backscatter tags forms a linear array.

Example 16: The system of example 14, wherein:
the at least four backscatter tags comprise:
   a first set of backscatter tags; and
   a second set of backscatter tags;
an arrangement of the first set of backscatter tags forms a first linear array along a first axis; and
an arrangement of the second set of backscatter tags forms a second linear array along a second axis that is approximately perpendicular to the first axis.

Example 17: The system of any one of examples 13-15, wherein a spacing between the at least two backscatter tags is greater than half a wavelength.

Example 18: The system of claim 17, wherein the spacing between the at least two backscatter tags is approximately equal to three-quarters of a wavelength.

Example 19: The system of any one of examples 13-18, wherein:
the reader comprises a radio-frequency identification reader; and
the at least two backscatter tags comprise at least two radio-frequency identification tags.

Example 20: The system of example 19, wherein the radio-frequency identification tags and the radio-frequency identification reader are configured according to an ultra-high frequency band or an extremely-high frequency band.

1. A method performed by a reader, the method comprising:
transmitting a radio-frequency signal using an antenna of the reader, the radio-frequency signal interacting with two backscatter tags to form two backscatter signals, the two backscatter signals reflected off of a person;
receiving the two backscatter signals reflected off of the person using the antenna; and
determining, based on the two backscatter signals, a location of the person and a vital sign of the person.

2. The method of claim 1, wherein:
the two backscatter signals are reflected off of another person; and
the determining comprises determining a location of the other person and a vital sign of the other person.

3. The method of claim 1, wherein,
the transmitting of the radio-frequency signal comprises transmitting the radio-frequency signal in a direction towards the two backscatter tags; and
the receiving of the two backscatter signals comprises receiving the two backscatter signals from a direction associated with an area of interest in which the person is located.

4. The method of claim 3, wherein the area of interest comprises:
a room in a building;
a bed;
a desk; or
a vehicle.

5. The method of claim 1, wherein:
the transmitting of the radio-frequency signal comprises transmitting the radio-frequency signal with a first polarization that is co-polarized with antennas of the backscatter tags; and
the receiving of the radio-frequency signal comprises receiving the radio-frequency signal with a second polarization that is cross-polarized with the antennas of the backscatter tags.

6. The method of claim 5, wherein:
the first polarization comprises a first circular polarization; and
the second polarization comprises a second circular polarization that is orthogonal to the first circular polarization.

7. The method of claim 6, wherein:
the first circular polarization comprises a left-hand circular polarization; and
the second circular polarization comprises a right-hand circular polarization.

8. The method of claim 1, wherein the vital sign comprises at least one of a breathing rate or a heart rate.

9. The method of claim 1, wherein:
the transmitting of the radio-frequency signal comprises transmitting the radio-frequency signal using a frequency-hopping spread-spectrum technique; and
the determining comprises calibrating input responses associated with the two backscatter signals to reduce variations caused by the frequency-hopping spread-spectrum technique.

10. A reader comprising at least one antenna, the reader configured to:
transmit a radio-frequency signal using the at least one antenna, the radio-frequency signal interacting with two backscatter tags to form two backscatter signals, the two backscatter signals reflected off of a person;
receive the two backscatter signals reflected off of the person using the at least one antenna; and
determine, based on the two backscatter signals, a location of the person and a vital sign of the person.

11. The reader of claim 10, wherein the at least one antenna comprises:
a transmit antenna; and
a receive antenna.

12. The reader of claim 10, wherein the at least one antenna is configured to transmit and receive radio-frequency signals with orthogonal circular polarizations.

13. A system comprising:
at least two backscatter tags; and
a reader comprising at least one antenna, the reader configured to:
transmit a radio-frequency signal using the at least one antenna, the radio-frequency signal interacting with the at least two backscatter tags to form at least two backscatter signals, the at least two backscatter signals reflected off of a person;
receive the at least two backscatter signals reflected off of the person using the at least one antenna; and
determine, based on the at least two backscatter signals, a location of the person and a vital sign of the person.

14. The system of claim 13, wherein the at least two backscatter tags comprise at least four backscatter tags.

15. The system of claim 14, wherein an arrangement of the at least four backscatter tags forms a linear array.

16. The system of claim 14, wherein:
the at least four backscatter tags comprise:
a first set of backscatter tags; and
a second set of backscatter tags;
an arrangement of the first set of backscatter tags forms a first linear array along a first axis; and
an arrangement of the second set of backscatter tags forms a second linear array along a second axis that is approximately perpendicular to the first axis.

17. The system of claim 13, wherein a spacing between the at least two backscatter tags is greater than half a wavelength.

18. The system of claim 17, wherein the spacing between the at least two backscatter tags is approximately equal to three-quarters of a wavelength.

19. The system of claim 13, wherein:
the reader comprises a radio-frequency identification reader; and
the at least two backscatter tags comprise at least two radio-frequency identification tags.

20. The system of claim 19, wherein the radio-frequency identification tags and the radio-frequency identification reader are configured according to an ultra-high frequency band or an extremely-high frequency band.
